(19) United States
(12) Patent Application Publication
(10) Pub. No.: US 2021/0090248 A1
(43) Pub. Date: Mar. 25, 2021

(54) CERVICAL CANCER DIAGNOSIS METHOD AND APPARATUS USING ARTIFICIAL INTELLIGENCE-BASED MEDICAL IMAGE ANALYSIS AND SOFTWARE PROGRAM THEREFOR

(71) Applicant: DoAI Inc., Seoul (KR)

(72) Inventors: Yong Jun CHOI, Seoul (KR); Hyun Gyu LEE, Pyeongtaek-si (KR); Bo Gyu PARK, Suwon-si (KR); Han Lim MOON, Seoul (KR)

(73) Assignee: DoAI Inc., Seoul (KR)

(21) Appl. No.: 16/725,625

(22) Filed: Dec. 23, 2019

Related U.S. Application Data
(63) Continuation of application No. PCT/KR2019/015215, filed on Nov. 11, 2019.

(30) Foreign Application Priority Data
Sep. 19, 2019 (KR) 10-2019-0115238

(51) Int. Cl. G06T 7/00; G16H 50/20; G16H 30/40; G06N 20/00; G06N 5/04; G06T 3/40

(52) U.S. Cl. CPC G06T 7/0012; G16H 50/20; G16H 30/40; G06N 20/00; G06T 2207/20132; G06T 3/40; G06T 2207/30024; G06T 2207/20081; G06N 5/04

(57) ABSTRACT

Provided is a method of diagnosing cervical cancer using an artificial intelligence-based medical image analysis, which is performed by a computer, the method including obtaining an image of cervical cells of an object; pre-processing the image; identifying one or more cells in the pre-processed image; determining whether the identified one or more cells are normal; and diagnosing whether the object has cervical cancer on the basis of a result of determining whether the identified one or more cells are normal.

### CROSS-REFERENCE TO RELATED APPLICATIONS

This application is a Continuation of International Application No. PCT/KR2019/015215 filed Nov. 11, 2019 which claims benefit of priority to Korean Patent Application No. 10-2019-0115238 filed Sep. 19, 2019, the entire content of which is incorporated herein by reference.

### TECHNICAL FIELD

The present disclosure relates to a cervical cancer diagnosis method and apparatus using an artificial intelligence-based medical image analysis, and a software program therefor.

### BACKGROUND ART

Cervical cancer is a major cause of death in women globally and is known to be caused by infection of human papillomavirus. Every year an average of 500,000 women are diagnosed with cervical cancer and 250,000 women die of cervical cancer. Human papillomavirus infection is most frequently found in women aged 20 to 24 years old (infection rate of 44.8%). Most human papillomavirus infections disappear naturally but may develop into cancer over 12 to 15 years when the infection passes into a chronic state.

The E6 and E7 proteins of human papillomavirus cause genetic instability and cell cycle perturbation of cervical epithelial cells, leading to deformation of the epithelial cells and to cancer. A Pap test is a diagnostic method that has been used for over 50 years to diagnose female uterine cell variants. When cell abnormality is found during the Pap test, colposcopy and biopsy are performed to specifically diagnose whether cancer has developed.

An early diagnosis of cervical cancer and vaccination therefor to prevent human papillomavirus infection have been recognized as the most important factors in reducing the incidence of cervical cancer, and the introduction of cytology using a Pap smear has contributed to significantly reducing the incidence of cervical cancer.

Various test methods using molecular diagnostic technology have been steadily proposed, as well as cell test methods such as the Pap test. It is known that an increase of an expression rate of Ki-67 and p16 in cells is closely related to cancerous uterine tissue. In addition, mini chromosome maintenance protein, cell division cycle protein 6, squamous cell carcinoma antigen and so on are known as major markers for a diagnosis of cervical cancer.

In addition, it has been known that a change of a sugar chain structure is closely related to the progress of a disease and the progress of cancer. Research results accumulated to date indicate that as cancer develops, sialylation and fucosylation increase at the surface of cancer cells and glycoconjugates in the blood.

Conventionally, there is a method of collecting and testing cells dropped from a patient's body to diagnose a disease which the patent is suffering from. Slides are manufactured by collecting samples of cells from the patient and performing Papanicolaou staining and encapsulating the slides, and are primarily inspected using an optical microscope by a screener (cytotechnologist). A slide considered as abnormal as a result of the primary inspection is secondarily deciphered by a pathologist to confirm a diagnosis of the lesion.

However, it takes a very long time for the screener to individually and manually inspect a large number of slides. Moreover, there is a limitation in manpower, because the number of qualified screeners is quite small and thus the number of skilled pathologists is very small.

In regions having a problem of the limitation in manpower, a method of applying a dilute acetic acid solution onto cervix to detect a part turned white, commonly known as the ‘Visual Inspection with Acetic acid (VIA)’ test method, is generally used. However, it has been generally evaluated that the VIA test method is inexpensive and easy to use but inaccurate.

In addition, because inspection depends on a pathologist's own experience and ability, human errors may occur according to the pathologist's condition during the inspection. To solve this problem, there have been field attempts to reduce errors by collecting primary inspection results and reviewing random samples but the cause of the problem cannot be structurally fixed.

In the background of this problem occurring in the field, there is a need for an electronic means for consistently and reliably inspecting multiple slides to provide a diagnostic result.

The importance of artificial intelligence technology used in the field of diagnostic radiology is greatly increasing. In modern medical science, medical imaging is a very important tool for effective diagnosis of diseases and treatment of patients. With the development of imaging technology, more accurate medical imaging data can be obtained and imaging technology is being continuously developed. Owing to sophisticated imaging technology, the amount of data is gradually increasing and thus there are difficulties in analyzing medical image data depending on human vision. Recently, clinical decision support systems and computer-assisted diagnostic systems are playing an essential role in an automatic medical image analysis.

In this technical background, the following technical idea will be provided herein.

### Disclosure

### Technical Problem

The present disclosure relates to a cervical cancer diagnosis method and apparatus using an artificial intelligence-based medical image analysis, and a software program therefor.

Aspects of the present disclosure are not limited thereto and other aspects not mentioned herein will be apparent to those of ordinary skill in the art from the following description.

### Technical Solution

To address the above-mentioned problems, a method of diagnosing cervical cancer using an artificial intelligence-based medical image analysis according to an aspect of the present disclosure includes obtaining an image of cervical cells of an object, pre-processing the image, identifying one or more cells in the pre-processed image, determining whether the identified one or more cells are normal, and diagnosing whether the object has cervical cancer, based on a result of determining whether the identified one or more cells are normal.

### Advantageous Effects

According to embodiments set forth herein, cervical cancer can be diagnosed on the basis of an artificial intelligence model even in an environment in which pathology specialists are insufficient.

In addition, a diagnosis method capable of preventing human errors which may occur in a diagnosis process and showing consistent accuracy can be provided.

Effects of the present disclosure are not limited thereto and other effects mentioned herein will be apparent to those of ordinary skill in the art from the following description.

### DESCRIPTION OF DRAWINGS

FIG. 1 is a diagram illustrating a system according to an embodiment.

FIG. 2 is a flowchart of a method of diagnosing cervical cancer using an artificial intelligence-based image analysis according to an embodiment.

FIG. 3 is a flowchart of a method of training an artificial intelligence model according to an embodiment.

FIG. 4 is a flowchart of an image pre-processing method according to an embodiment.

FIG. 5 is a flowchart of a method of training an artificial intelligence model according to a resolution according to an embodiment.

FIG. 6 is a flowchart of an image quality management method according to an embodiment.

FIG. 7 is a flowchart of a diagnosis method according to an embodiment.

FIG. 8 is a flowchart of a High-grade Squamous Intraepithelial Lesion (HSIL) classification method according to an embodiment.

FIGS. 9 and 10 are diagrams illustrating examples of determining whether a cell is normal by identification and classification of an image of the cell.

FIG. 11 is a diagram illustrating an example of an annotation task.

FIG. 12 illustrates an image of a plurality of cells.

FIG. 13 illustrates a training process performed based on a result of identifying a plurality of regions, and normal and abnormal cells detected as a result of the training process according to an embodiment.

FIG. 14 is a block diagram of an apparatus according to an embodiment.

### BEST MODE

According to one aspect of the present disclosure, a method of diagnosing cervical cancer using an artificial intelligence-based medical image analysis includes obtaining an image of cervical cells of an object (S110), pre-processing the image (S120), identifying one or more cells in the pre-processed image (S130), determining whether the identified one or more cells are normal (S140), and diagnosing whether the object has cervical cancer based on a result of the determining in operation S140 (S150).

In operations S130 and S140, one or more cells in the pre-processed image are identified and whether the identified one or more cells are normal are determined using a pre-trained artificial intelligence model.

The method may further include obtaining training data including one or more cervical cell images (S210), pre-processing the images included in the training data (S220), and training the artificial intelligent model using the images pre-processed in operation S220 (S230).

Operation S220 may include resizing the images included in the training data (S310), adjusting colors of the resized images (S320), deriving a contour of each of the color-adjusted images (S330), and cropping the images on the basis of the contours derived in operation S250 (S340).

Operation S230 may include obtaining a pre-processed high-resolution image and a pre-processed low-resolution image (S410), training a first model using the high-resolution image (S420), training a second model using the low-resolution image (S430) and assembling results of training the first model and the second model (S440).

Operation S110 may include determining suitability of the obtained image (S510) and requesting to obtain an image again on the basis of the determined suitability (S520). The requesting of the obtaining of the image again may include at least one of requesting to capture an image again or requesting to obtain a sample again.

Operation S140 may include classifying the identified one or more cells into at least one of categories including normal, Atypical Squamous Cells of Undetermined Significance (ASCUS), Atypical Squamous Cells, cannot exclude HSIL (ASCH), Low-grade Squamous Intraepithelial Lesion (LSIL), High-grade Squamous Intraepithelial Lesion (HSIL), or a cancer (S610). Operation S150 may include counting the number of cells classified in each of the categories in operation S610 (S620), assigning weights to the categories (S630), calculating cervical cancer diagnosis scores on the basis of the weight and the number of counted cells for each of the categories (S640), and diagnosing whether the object has cervical cancer on the basis of the scores (S650).

Operation S610 may include identifying nucleus and cytoplasm of each of the identified cells (S710), calculating areas of the identified nucleus and cytoplasm (S720), and calculating an HSIL score of each of the identified cells on the basis of the ratio between the areas of the nucleus and cytoplasm (S730).

According to another aspect of the present disclosure, an apparatus includes a memory storing one or more instructions and a processor for executing the one or more instructions stored in the memory, wherein the processor may execute the one or more instructions to perform a cervical cancer diagnosis method using an artificial intelligence-based medical image analysis.

According to another aspect of the present disclosure, there is provided a computer program stored in a computer-readable recordable medium combined with a computer which is a hardware component to perform a cervical cancer diagnosis method using an artificial intelligence-based medical image analysis.

Other details of the present disclosure are provided in the detailed description and drawings.

### MODES OF THE INVENTION

Advantages and features of the present disclosure and methods of achieving them will be apparent from embodiments described in detail in conjunction with the accompanying drawings. However, the present disclosure is not limited to embodiments set forth herein and may be embodied in many different forms. The embodiments are merely provided so that this disclosure will be thorough and complete and will fully convey the scope of the disclosure to those of ordinary skill in the art. The present disclosure should be defined by the claims.

The terms used herein are for the purpose of describing embodiments only and are not intended to be limiting of the present disclosure. As used herein, singular forms are intended to include plural forms unless the context clearly indicates otherwise. As used herein, the terms “comprise” and/or “comprising” specify the presence of stated components but do not preclude the presence or addition of one or more other components. Throughout the disclosure, like reference numerals refer to like elements, and “and/or” includes each and all combinations of one or more of the mentioned components. Although “first”, “second”, etc. are used to describe various components, these components are not limited by these terms. These terms are only used to distinguish one component from another. Therefore, a first component discussed below could be termed a second component without departing from the technical scope of the present disclosure.

Unless otherwise defined, all terms (including technical and scientific terms) used herein have the same meaning as commonly understood by one of ordinary skill in the art to which the present disclosure pertains. Terms such as those defined in commonly used dictionaries will not be interpreted in an idealized or overly formal sense unless expressly so defined herein.

The term “unit” or “module” used herein should be understood as software or a hardware component, such as a field-programmable gate array (FPGA) or an application-specific integrated circuit (ASIC), which performs certain functions. However, the term “unit” or “module” is not limited to software or hardware. The term “unit” or “module” may be configured to be stored in an addressable storage medium or to reproduce one or more processors. Thus, the term “unit” or “module” should be understood to include, for example, components such as software components, object-oriented software components, class components, and task components, processes, functions, attributes, procedures, subroutines, segments of program code, drivers, firmware, microcode, a circuit, data, database, data structures, tables, arrays, and parameters. Functions provided in components and “units” or “modules” may be combined into smaller numbers of components and “units” or “modules” or divided into subcomponents and “subunits” or “submodules”.

Spatially relative terms, such as “below”, “beneath”, “lower”, “above”, “upper” and the like, may be used herein for ease of description of the relationship between one element and other elements as illustrated in the drawings. Spatially relative terms are intended to encompass different orientations of components in use or operation in addition to the orientations depicted in the drawings. For example, when one component illustrated in each of the drawings is turned upside down, another component referred to as “below” or “beneath” the component may be located “above” the component. Thus, the illustrative term “below” should be understood to encompass both an upward direction and a downward direction. Components can be oriented in different directions as well and thus spatially relative terms can be interpreted according to orientation.

In the present specification, the term “computer” refers to all types of hardware devices that each include at least one processor and may be understood to include a software configuration operated in a hardware device according to an embodiment. For example, a computer may be understood to include, but is not limited to, a smartphone, a tablet PC, a desktop computer, a notebook computer, and a user client and an application running in each device.

Hereinafter, embodiments of the present disclosure will be described in detail with reference to the accompanying drawings.

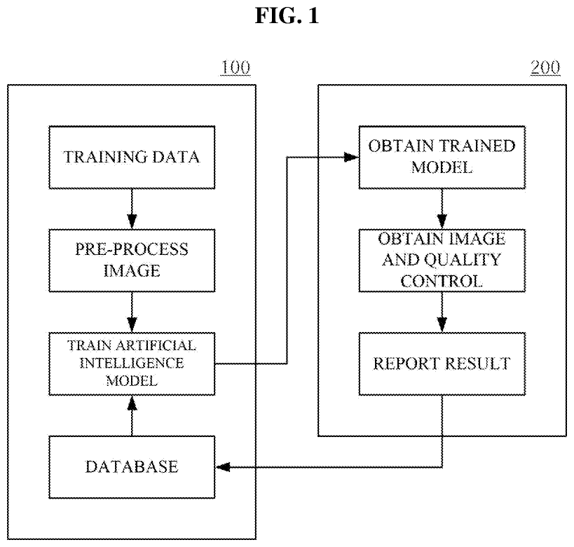

FIG. 1 is a diagram illustrating a system according to an embodiment.

FIG. 1 illustrates a server 100 and a user terminal 200.

In the present embodiment, the server 100 may be a type of computer described above but is not limited thereto. For example, the server 100 may refer to a cloud server.

The user terminal 200 may also be a type of computer described above and may refer to, for example, a smart phone, but is not limited thereto.

In one embodiment, the server 100 may train an artificial intelligence model for performing a cervical cancer diagnosis method using an artificial intelligence-based image analysis according to an embodiment set forth herein.

In addition, the user terminal 200 may perform a cervical cancer diagnosis method using an artificial intelligence-based image analysis using an artificial intelligence model trained via the server 100 according to an embodiment set forth herein.

However, the server 100 and the user terminal 200 are not limited thereto, and at least a part of an artificial intelligence model training method may be performed by the user terminal 200, and the server 100 which obtains information from the user terminal 200 may perform a cervical cancer diagnosis through an image analysis using an artificial intelligence model and transmit a diagnosis result to the user terminal 200.

In one embodiment, the server 100 may obtain training data. The training data may include, but is not limited to, an image of cervical cells, and specifically, an image of a result obtained by smearing the cervical cells on a slide for Pap smear test and performing necessary processing such as staining and the like.

The server 100 may pre-process images included in the training data. A method of pre-processing the images will be described in detail later.

The server 100 may train the artificial intelligence model using the pre-processed images. In an embodiment set forth herein, the artificial intelligence model may refer to a model trained based on machine learning technology but is not limited thereto. Although a type of machine learning technology and an algorithm thereof are not specifically limited, deep learning technology may be used, and more specifically, Mask R-CNN technology may be used.

As another example, a lightweight model based on SSDlite and Mobilenet v2 may be used but embodiments are not limited thereto.

In addition, the user terminal 200 may obtain the artificial intelligence model trained by the server 100. In one embodiment, the user terminal 200 may obtain one or more parameters corresponding to a result of training the artificial intelligence model by the server 100.

In an embodiment set forth herein, the user terminal 200 may perform a method according to the embodiment using an application installed therein but embodiments are not limited thereto.

For example, the method according to the embodiment may be provided on a web basis, not on an application, or may be provided on a system basis such as a picture archiving and communication system (PACS).

Services based on such a system may be provided embedded in a specific device but may be provided remotely via a network, or at least some functions may be provided distributed to different devices.

As another example, the method according to the embodiment may be provided based on a software as a service (SaaS) or other cloud-based systems.

All techniques, methods, and operations provided herein are not limited to being provided based on a specific subject or system as described above.

In one embodiment, a trained model used in the user terminal 200 may refer to a lightweight model appropriate for the performance of the user terminal 200.

In one embodiment, the user terminal 200 may obtain an image of cervical cells of an object. The user terminal 200 may provide feedback for management of the quality of the obtained image as will be described in detail later.

As used herein, the term “object” should be understood to include a human being or animal or a part thereof. For example, the object may include an organ, such as a liver, heart, uterus, brain, breast, or abdomen, or blood vessels.

Examples of a “user” may include, but are not limited to, a medical professional, such as a doctor, a nurse, a clinical pathologist, or a medical image expert, and a technician who repairs medical devices. For example, a user may refer to a manager who performs a medical examination using a system according to an embodiment in a medically vulnerable region or a patient.

The user terminal 200 may analyze an obtained image on the basis of an artificial intelligence model and diagnose whether an object has cervical cancer on the basis of a result of analyzing the image.

The user terminal 200 may report an examination result to a user and transmit feedback on the examination result to the server 100. The server 100 may store the feedback in a database and retrain and update the artificial intelligence model on the basis of the feedback.

Operations included in a method of diagnosing cervical cancer using an artificial intelligence-based image analysis according to an embodiment will be described in detail with reference to the accompanying drawings below.

Operations described below will be described as being performed by a computer, but a subject of each of the operations is not limited thereto and at least some of the operations may be performed by different devices according to an embodiment.

For example, the computer may include at least one of the server 100 or the user terminal 200 but embodiments are not limited thereto.

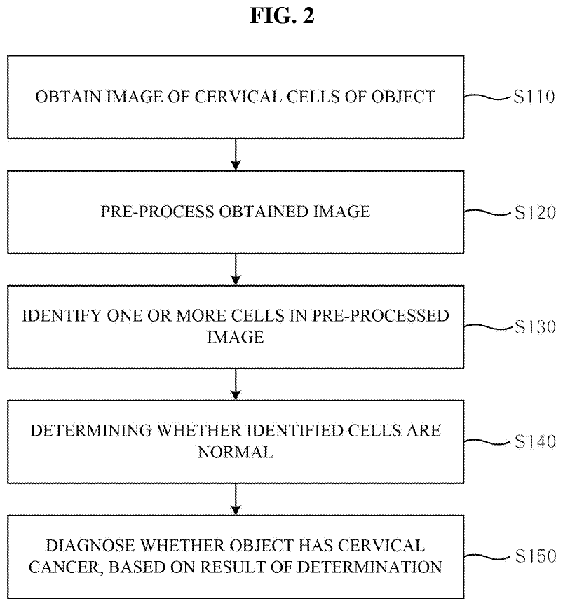

FIG. 2 is a flowchart of a method of diagnosing cervical cancer using an artificial intelligence-based image analysis according to an embodiment.

In operation S110, a computer obtains an image of cervical cells of an object.

In one embodiment, the computer may obtain an image of a slide smeared with cervical cells of the object that is captured by a smartphone camera. In one embodiment, a camera different from a smartphone camera may be used.

The slide smeared with the cervical cells of the object may refer to a result of performing operations, e.g., staining after cell smearing, which are necessary for a Pap smear test.

In an embodiment set forth herein, a method of smearing a slide with cells and performing pre-processing thereon may include, but is not limited to, a method based on a conventional Pap smear method or a method based on a liquid-based cytology method. That is, an analysis of an image of cells and a cervical cancer diagnosis method based thereon are not limited to the method of smearing a slide with cells and performing pre-processing thereon, and various artificial intelligence models trained on the basis of training data collected based on different pre-processing methods may be used.

In one embodiment, an artificial intelligence model may be trained by synthesizing training data collected on the basis of different smearing and pre-processing methods so that the artificial intelligence model may be trained to diagnose cervical cancer of an object regardless of a smearing and pre-processing method.

In one embodiment, an artificial intelligence model trained to diagnose cervical cancer of an object regardless of a smearing and pre-processing method is provided and different artificial intelligence models that are finely tuned according to training data collected based on different smearing and pre-processing methods may be provided so that an artificial intelligence model showing higher accuracy with respect to different smearing and pretreatment methods may be obtained and used, but embodiments are not limited thereto.

In one embodiment, auxiliary equipment, such as a magnifying glass, a lens, and a microscope, attached to a camera or provided separately from the camera may be used, and an image enlarged by the auxiliary equipment may be captured by the camera.

When an image is captured based on a smartphone application, data associated with the smartphone application may be stored and managed together with the image and used in the future to diagnose cervical cancer according to an embodiment set forth herein together with a result of analyzing the image.

For example, when an image is captured using a smartphone application, information regarding a patient (object) corresponding to the image may be input together with the image or obtained in various ways, and stored together with the image to be used for an analysis of the image or used to determine whether the object has cervical cancer together with a result of analyzing the image.

In addition, an identifier (ID) of the patient (object) may be created and labeled in image data on the basis of the smartphone application and used to match information regarding the patient or to store information regarding the patient.

In operation S120, the computer pre-processes the image.

In one embodiment, pre-processing performed at an examination stage may be the same as pre-processing performed in a learning stage to be described later or may include at least a part of the pre-processing performed in the learning stage.

In one embodiment, an annotation task may be performed by a user in the pre-processing.

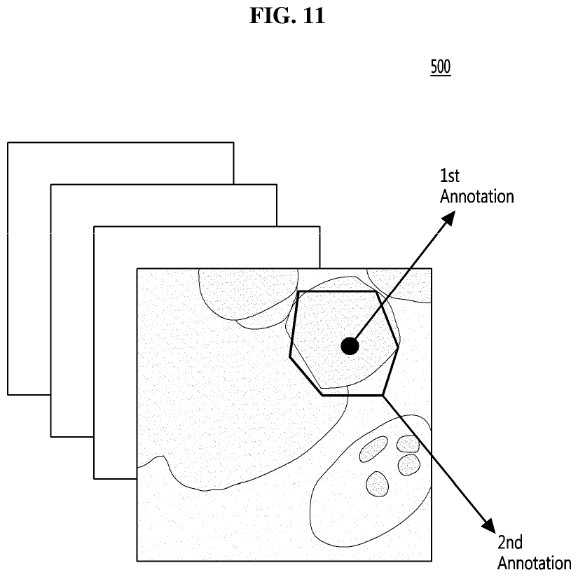

FIG. 11 illustrates an example 500 of an annotation task.

At the examination stage, the annotation task may include a task of designating a region including a cell or selecting a central point on the cell using an input means, such as a touch input, a touch pen, or a mouse, in an image displayed on a screen of a user terminal.

In one embodiment, the annotation task may include, but is not limited to, primary annotation for selecting the central point on the cell and secondary annotation for selecting or inputting a region including the cell.

In one embodiment, a bounding box for the cell or nucleus may be generated on the basis of the annotation task.

In operation S130, the computer identifies one or more cells in the pre-processed image.

In one embodiment, the computer may identify one or more cells in an image using a trained artificial intelligence model. For example, the computer may identify a region including cells in the images and further identify a region including cell membrane, cytoplasm and nucleus, but embodiments are not limited thereto.

A pre-trained Mask R-CNN model may be used for identification of cells as described above but embodiments are not limited thereto.

As another example, a lightweight model based on SSDlite and Mobilenet v2 may be used but embodiments are not limited thereto.

In operation S140, the computer identifies whether the identified one or more cells are normal.

In one embodiment, the computer may identify whether the identified one or more cells are normal or include specific abnormality. In one embodiment, the computer may classify the cells into at least one of a normal state or an abnormal state including one or more categories. For the classification of the cells, the pre-trained Mask R-CNN model may be used as described above but embodiments are not limited thereto.

As another example, a lightweight model based on SSDlite and Mobilenet v2 may be used but embodiments are not limited thereto.

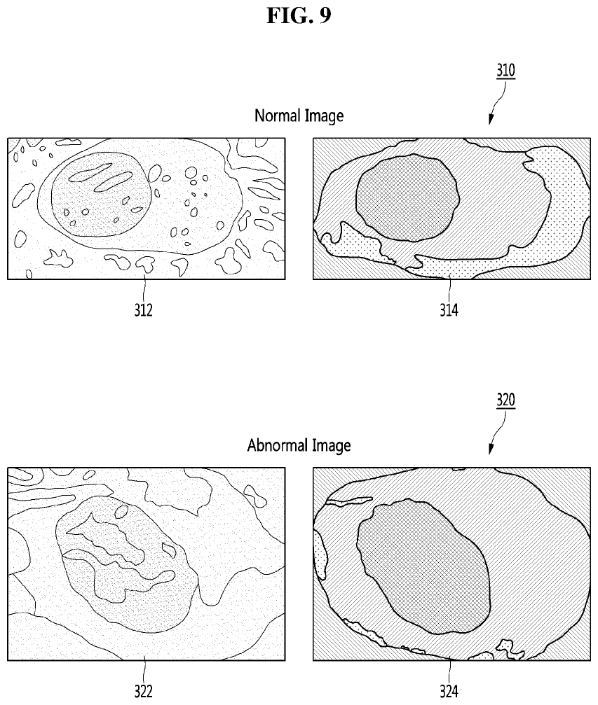

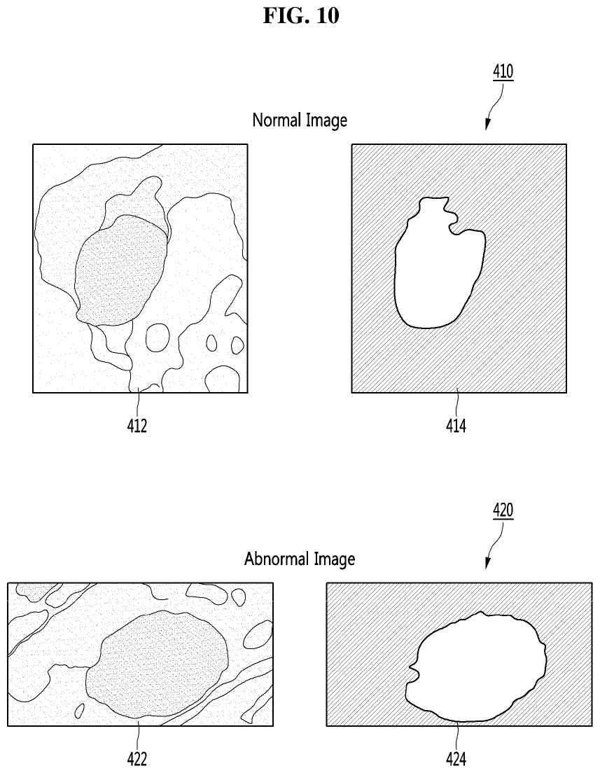

FIGS. 9 and 10 illustrate examples of determining whether a cell is normal by identification and classification of an image of the cell.

FIG. 9 illustrates a normal case 310 and an abnormal case 320.

In one embodiment, although the drawing is shown in black and white, cytoplasm and a cell membrane of a normal cell are relatively large and may be blue or pink due to staining. However, an example of a criterion for identifying a normal cell is provided here and thus a criterion for identifying normal and abnormal cells is not limited thereto. In addition, the identifying of normal and abnormal cells may be performed on the basis of other various criteria which are not set in a process of training an artificial intelligence model.

In the case of the normal case 310, a cell may be determined to be normal on the basis of an image 314 obtained by identifying nucleus, cytoplasm, and cell membrane from an image 312 of the cell.

Similarly, in case of the abnormal case 320, a cell may be determined to be abnormal on the basis of an image 324 obtained by identifying a nucleus, cytoplasm, and cell membrane from an image 322 of the cell.

FIG. 10 illustrates a normal case 410 and an abnormal case 420.

In the case of the normal case 410, a cell may be determined to be normal on the basis of an image 414 obtained by identifying a region corresponding to nucleus from an image 412 of the cell. In this case, in at least some of learning and examination operations, a pre-processing process may be performed in which a nucleus is identified and pre-processed and cytoplasm and the cell membrane are identified and excluded. For example, the nucleus may be identified by color and pre-processed and the cytoplasm and the cell membrane may be identified by color and excluded.

Alternatively, pre-processing may be performed by bending lines of features identified on the basis of an elastic transform.

An artificial intelligence model may be trained based on an image pre-processed as described above, and whether each cell is abnormal may be identified when a diagnosis is performed based on the artificial intelligence model by analyzing a raw image or an image, at least some of which has been pre-processed using the artificial intelligence model.

As such, in the case of the abnormal case 440, a cell may be determined to be abnormal on the basis of an image 424 obtained by identifying a region corresponding to a nucleus from an image 422 of the cell.

In operation S150, the computer diagnoses whether the object has cervical cancer on the basis of a result of the determining in operation S140.

In one embodiment, the computer may diagnose cervical cancer of the object on the basis of the type and number of cells determined as abnormal but embodiments are not limited thereto.

In one embodiment, the computer may calculate a cervical cancer diagnostic score of the object or calculate a degree of risk, the likelihood of occurrence, or the like. The computer may suggest a subsequent procedure to a user on the basis of a result of the calculation. For example, when a diagnosis result indicating the possibility of cervical cancer is obtained, the computer may recommend the user receive hospital treatment, a remote medical service, re-examination, a complete medical examination, or the like.

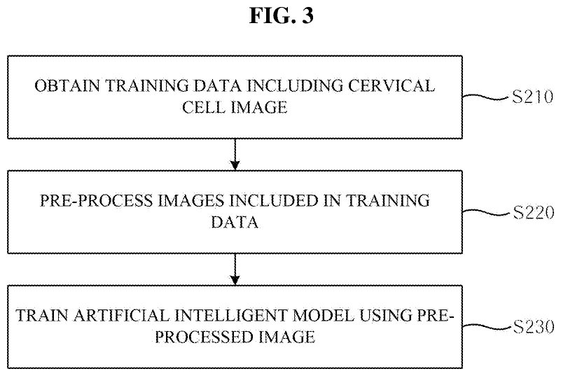

FIG. 3 is a flowchart of a method of training an artificial intelligence model according to an embodiment.

In operation S210, the computer may obtain training data including one or more cervical cell images.

In one embodiment, the training data may include, but is not limited to, a cervical cell image and an image of a result obtained by smearing a slide with the cervical cells and performing necessary processing, such as staining, thereon for a Pap smear test.

The training data may further include labeling information on whether each of the cervical cell images represents normal or abnormal. Whether each of the cervical cell images represents normal or abnormal may be diagnosed directly by a pathologist. The training data may further include information obtained by determining whether each of the cervical cell images represents normal or abnormal by one or more other test methods other than the Pap smear.

The training data may further include classification information regarding a category to which a cell belongs when the cell is an abnormal cell. Types of abnormal categories will be described later.

The artificial intelligence model trained based on the training data may identify whether each of the cervical cell images is normal or abnormal and identify information regarding a category to which a cervical cell belongs when the cervical cell image thereof is abnormal. In one embodiment, the artificial intelligence model may calculate a probability that a cell belongs to each category.

In addition, the training data may further include an image including a plurality of cervical cells and include labeling information on whether each of the cells included in the image is normal or abnormal, and in addition, information as to whether an object corresponding to the image has been diagnosed with cervical cancer. Furthermore, the training data may further include information as to whether an object corresponding to each image has developed into cervical cancer after a certain time period although the object was not diagnosed with cervical cancer when each image was captured, information regarding a treatment method of the cervical cancer, and information regarding prognosis of the cervical cancer.

An artificial intelligence model trained based on the training data is capable of identifying whether each cell is normal or abnormal, estimating whether an object has cervical cancer on the basis of an image including a plurality of cells, and predicting whether there is a risk of cervical cancer or whether cervical cancer may occur at a certain point in time even when a corresponding object does not have cervical cancer at a current point in time.

In addition, the artificial intelligence model is capable of predicting a treatment method and prognosis when cervical cancer occurs in each object and recommending information regarding improvement of living conditions for prevention of cervical cancer, drug treatment, surgical treatment or a follow-up on the basis of the predicted treatment method and prognosis.

In addition, the artificial intelligence model may determine the probability of metastasis or the risk of metastasis when cervical cancer occurs in each object and provide information regarding one or more treatment methods for prevention of metastasis.

In operation S220, the computer may pre-process images included in the training data. A method of pre-processing the images will be described in detail later.

In operation S230, the computer may train the artificial intelligence model using the images pre-processed in operation S220. A method of training the artificial intelligence model on the basis of the images is not limited but, for example, a deep learning technique based on a convolutional neural network (CNN) may be used. More specifically, the R-CNN technique may be used, and in particular, the Mask R-CNN technique may be used, but embodiments are not limited thereto.

The R-CNN technique may suggest a plurality of region proposals and include a method of analyzing an image through operations, such as feature extraction and classification, by analyzing each region on the basis of the CNN.

As another example, a lightweight model based on SSDlite and Mobilenet v2 may be used but embodiments are not limited thereto.

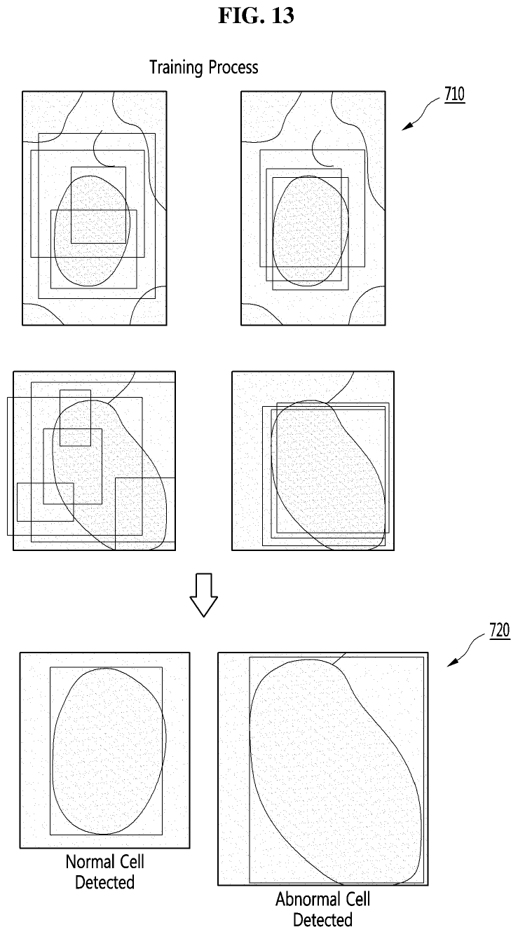

FIG. 13 illustrates a training process 710 performed based on a result of identifying a plurality of regions, and normal and abnormal cells 720 detected as a result of the training process 710.

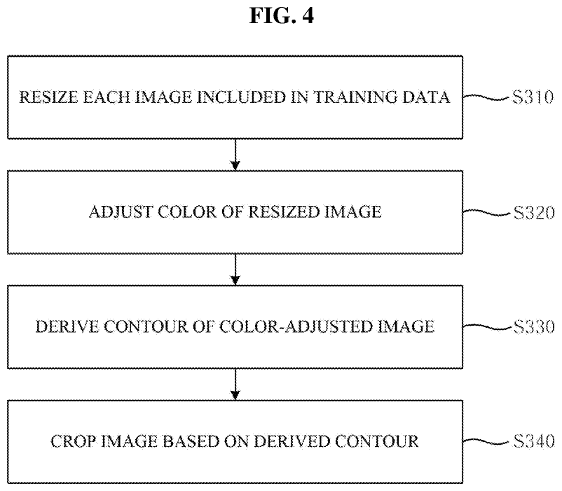

FIG. 4 is a flowchart of an image pre-processing method according to an embodiment.

A pre-processing method described below may be used to not only process training data for training an artificial intelligence model but also pre-process an image to be diagnosed at a diagnosis stage using the trained artificial intelligence model.

In a pre-processing operation according to an embodiment set forth herein, the annotation task described above may be performed. A computer may obtain a bounding box for a cell region or a nucleus region on the basis of the annotation task and perform an analysis on the basis of the annotation task.

In operation S220 described above, the computer may resize the images included in the training data (S310).

In one embodiment, the computer may downsize the images after upscaling the images, and a method and sequence for scaling the image are not limited.

In one embodiment, the computer may obtain images having different resolutions by performing dilated convolution on an image in a network-based learning process and upscale the images to have an original resolution.

In one embodiment, the computer may not use pooling when an image of a cell is below a predetermined criterion.

In addition, the computer may adjust colors of the resized images (S320).

In one embodiment, a cell included in an image may be stained after smearing. Accordingly, the computer may adjust colors of the image to clearly differentiate between colors of stained nucleus, cytoplasm, cell membrane, and other regions. A method of adjusting the colors of the image is not limited but color adjustment may be performed using a filter for adjusting brightness or chroma but embodiments are not limited thereto.

In one embodiment, colors of an image may be adjusted differently according to a state of the image. For example, a color processing method required may vary according to whether cell membrane or cytoplasm will be highlighted to be identified or whether nucleus will be highlighted to be identified.

As an unrestrictive example, at a learning or diagnosis stage, colors may be adjusted to highlight cytoplasm and membrane so as to identify whether a cell is normal. When the cell is identified to be abnormal at the at a learning or diagnosis stage, colors may be readjusted to highlight a nucleus so as to obtain a nucleus region and a feature of the nucleus region may be analyzed to determine whether the nucleus region is abnormal and determine an abnormal category.

By highlighting the colors of the nucleus, the cytoplasm, and the cell membrane, the computer is capable of identifying shapes and boundaries of the regions thereof more accurately.

In one embodiment, the adjusting of the color of each resized image may include binarization of each image. For example, as illustrated in FIG. 10, a nucleus and remaining regions may be binarized and displayed for identification of the shape of the nucleus.

In addition, the computer may derive a contour of each of the color-adjusted images (S330).

For example, the computer may obtain boundaries of a nucleus, cytoplasm and a cell membrane on the basis of the differences between the colors of each of the images and generate training data on the basis of the boundaries.

In one embodiment, the computer may separate images of a nucleus, cytoplasm, and a cell membrane included in each of the images on the basis of the contours and train different artificial intelligence models on the basis of shapes of the cell nucleus, the cytoplasm, and the cell membrane. Each of the trained different artificial intelligence models is capable of identifying whether each of the nucleus, the cytoplasm, and the membrane is abnormal on the basis of the shapes thereof. In addition, the computer may assemble the trained different artificial intelligence models and compare results of the assembling with each other to determine whether each cell is abnormal and to obtain information regarding a category to which each cell belongs.

In addition, the computer may crop the images on the basis of the contours derived in the operation S250 (S340).

For example, the computer may crop the images on the basis of the obtained bounding box and train an artificial intelligence model on the basis of the cropped images. In one embodiment, a size or resolution of an image to be input to the artificial intelligence model may be limited or fixed. In this case, the computer may crop the images according to size or resolution and use techniques such as upscaling or downsizing to adjust the resolution or the size.

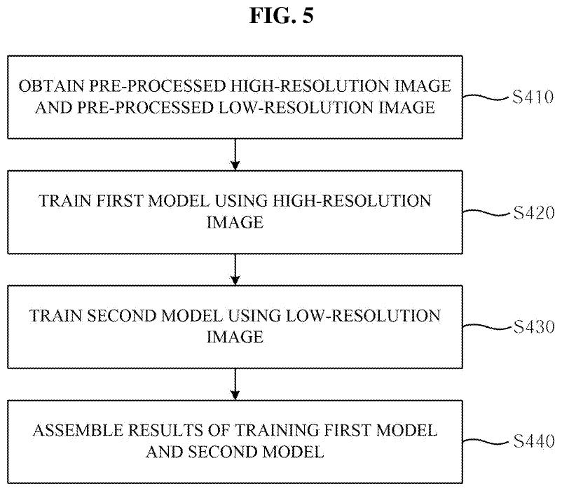

FIG. 5 is a flowchart of a method of training an artificial intelligence model according to a resolution according to an embodiment.

In operation S230 described above, the computer may obtain a pre-processed high-resolution image and a pre-processed low-resolution image (S410).

For example, the computer may obtain images having various resolutions by performing techniques, such as upscaling, downsizing, cropping and dilated convolution, on an image. The images having different resolutions may have different features and thus the computers may perform learning and diagnosing on the basis of high-resolution features (fine features) and low-resolution features (coarse features) of the images and assemble results of performing learning and diagnosing.

In addition, the computer may obtain images having various resolutions using a technique such as dilated convolution and upscale the images to have an original resolution.

In addition, the computer may train a first model using the high-resolution image (S420).

For example, the first model may use a Residential Energy Services Network (Resnet) 101 as a backbone network but embodiments are not limited thereto.

In addition, the computer may train a second model using the low-resolution image (S430).

For example, the second model may use Resnet 50 as a backbone network but embodiments are not limited thereto.

The number of layers of each of the Resnets described above is not limited thereto and may be adjusted differently on the basis of the resolution of each of the images and a result of processing each of the images.

In addition, the computer may assemble results of training the first model and the second model (S440).

The above-described backbone networks may use a method of separately learning a cell of a completely normal part and a cell of an ambiguous part and ensembling results of the learning.

In one embodiment, the cell of the completely normal part and the cell of the ambiguous part may be learned by applying different pre-processing methods including color adjustment thereto, and the cell of the ambiguous part may be diagnosed more accurately by performing learning using a plurality of different pre-processing methods and assembling results of the learning.

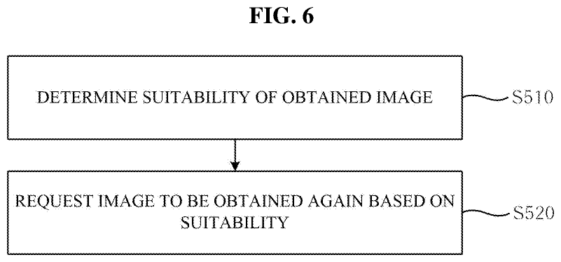

FIG. 6 is a flowchart of an image quality management method according to an embodiment.

In operation S110, the computer may identify suitability of the obtained image (S510).

For example, the computer may identify that a captured image is not suitable when the resolution of the captured image is less than or equal to a predetermined level or when an indicator which can be quantitatively evaluated, e.g., light reflection or blurring, is beyond a predetermined range.

The computer may request to obtain an image again on the basis of the identified suitability (S520).

For example, the computer may request to capture an image again until a predetermined criterion is satisfied.

In one embodiment, the computer may analyze features of an image, identify one or more causes of unsuitability of the image, and provide the one or more causes to a user. In addition, the computer may suggest a photographing method to the user for improving the one or more causes of unsuitability of the image. For example, the computer may suggest focus adjustment during a photographing process when a resolution of the image is low and may request to clean a lens of a camera or microscope when the image is blurry. When there is light reflection, the computer may request to remove a light source in a corresponding direction, remove light using a screen, or change a photographing direction.

The requesting of the obtaining of the image again may include at least one of requesting to capture an image again or requesting to obtain a sample again according to a state of the image.

For example, an analysis of the image may reveal not only unsuitability of the image, such as low resolution, blurring or light reflection, occurring in the photographing process but also problems with processing, such as cell smearing, preservation, and staining.

A diagnostic method according to the embodiments set forth herein may be subject to capturing an image on the basis of a sample obtained and processed according to a manual in an environment in which the number of medical professionals is insufficient and thus an evaluation of the sample is also determined as a necessary step.

For example, a plurality of overlapping cells due to insufficient and uneven smearing of cells at a smearing stage may be identified.

In one embodiment, a method of smearing cells on a glass slide using a cotton swab may be used, but in this case, some cells may be clustered in multiple layers and a non-uniform result having no cells at a particular location may be obtained. In order to overcome this problem, recently, a method of obtaining only epithelial cells by centrifugation, such as liquid cytodiagnosis, and evenly smearing the epithelial cells on a glass plate may be used but such equipment and technique may be difficult to use in an environment such as that described in the embodiments set forth herein.

Thus, the computer may identify a smeared state of the cells and request to perform processing or obtain a sample again according to a result of the identification.

For example, the computer may identify overlapping of cells or components (cell nucleus, cytoplasm and membrane) of the cells during identification of the cells and the components. For example, when the difference between colors of the inside of a region classified as a nucleus in color-based classification is greater than or equal to a predetermined level, it may be determined that the color difference occurs due to overlapping of a plurality of nuclei.

When overlapping of cells is suspected, the computer may identify the cells and components within a certain range of surroundings of the cells, set a contour, separate the components from each other, highlight the components by color adjustment, and analyze the difference between colors of the insides of the components or shapes of the components on the basis of the contour. It may be determined that a plurality of components overlap each other when the difference between the colors of the insides of the components is greater than or equal to the predetermined level or when the shapes of the components do not meet a predetermined criteria (for example, when the shapes of the components are not round, elliptical, or the like or when it is determined that a change of an angle of the set contour is greater than or equal to a predetermined level).

The computer may exclude the overlapping cells so as not to be identified and count the number of non-overlapping cells. When the number of the non-overlapping cells is less than or equal to a predetermined reference value, the computer may determine that the sample is difficult to test and thus request the user to obtain a sample again.

When an input informing that a sample cannot be obtained again is received, the computer may identify whether one or more non-overlapping cells included in the sample are normal or abnormal and identify whether one or more cells considered as overlapping each other are normal or abnormal. However, the computer may calculate an overall diagnosis result by assigning lower weights to whether the cells considered as overlapping each other are normal or abnormal and the categories thereof than the non-overlapping cells, thereby obtaining as high a diagnosis result as possible using a limited sample. In addition, a weight may be set differently according to the number of the overlapping cells and may be set to be lower, for example, as the number of the overlapping cells increases.

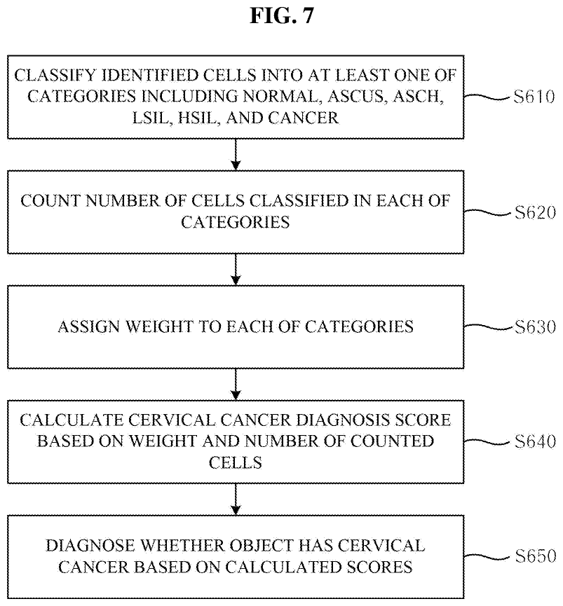

FIG. 7 is a flowchart of a diagnostic method according to an embodiment.

In operation S140 described above, a computer may classify the identified one or more cells into at least one of categories, including normal, Atypical Squamous Cells of Undetermined Significance (ASCUS), Atypical Squamous Cells, cannot exclude HSIL (ASCH), Low-grade Squamous Intraepithelial Lesion (LSIL), High-grade Squamous Intraepithelial Lesion (HSIL), or a cancer (S610).

The types of categories described above are not limited thereto, and at least some thereof may be excluded or other categories not described herein may be further added.

In operation S150, the computer may count the number of cells classified into each of the categories in operation S610 (S620).

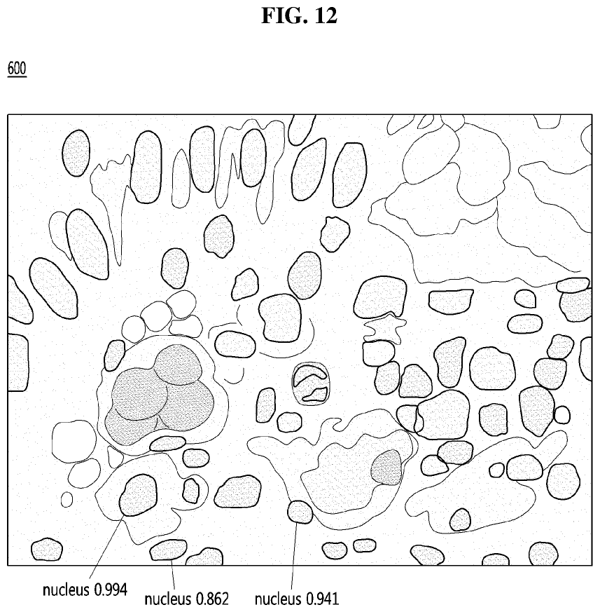

FIG. 12 illustrates an image 600 including a plurality of cells.

Although FIG. 12 illustrates the image 600 including a plurality of cells, the image 600 of FIG. 12 is provided as an example and smearing and pre-processing methods used in the methods according to the embodiments set forth herein and the type of an image obtained thereby are not limited. For example, not only an image obtained based on the aforementioned conventional Pap smear method but also an image obtained based on liquid-based cytology may be used, and a method of smearing various types of cells which do not meet a predetermined rule according to an environment and an image based thereon may be used.

In addition, the computer may assign a weight to each of the categories (S630).

For example, different weights may be assigned to the categories on the basis of a progress rate, e.g., a cancer progress rate of 20% in the case of the ASCUS and a cancer progress rate of 30% in the case of HSIL, a cancer incidence rate, and a degree of risk.

For example, different probabilities may be given to the categories according to a cancer progress rate, and a final cancer incidence probability may be calculated by multiplying a result of the counting by each of the probabilities.

In addition, the computer may calculate a cervical cancer diagnosis score on the basis of the weights and the number of counted cells for each of the categories (S640).

For example, a cancer incidence probability (or diagnostic score) may be calculated by dividing the sum of the products of the numbers of cells counted for the categories and cancer progress rates corresponding to the categories by the total number of counted cells.

In addition, the computer may diagnose whether the object has cervical cancer on the basis of the calculated score (S650).

For example, the computer may determine whether a cancer develops according to a range of the calculated probability (diagnosis score) or recommend a countermeasure therefor. For example, the computer may provide a result, such as re-examination, complete medical examination, a physician's care, or telemedicine, according to the range of the calculated probability. In one embodiment, telemedicine may refer to a procedure for transmitting image data to a server through which the image data may be checked by a medical specialist and obtaining a result of the checking when it is difficult to identify the result.

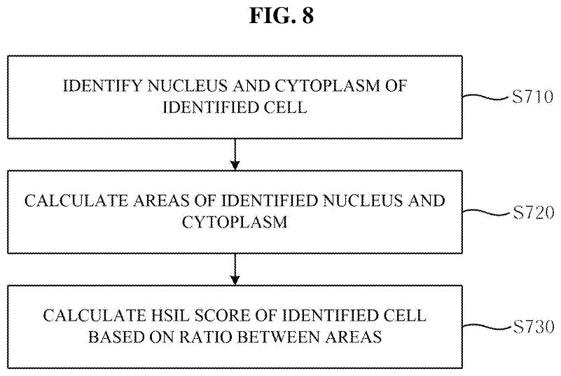

FIG. 8 is a flowchart of an HSIL classification method according to an embodiment.

For example, in the case of the HSIL (abnormal) category, a criterion of determination may be determined according to the ratio of the areas occupied by components of a cell. For example, the areas of cytoplasm and nucleus may be calculated, and a higher probability may be given to the HSIL category as the difference between the two areas decreases.

To this end, in operation S610 described above, the computer may identify a nucleus and cytoplasm of each of the identified one or more cells (S710).

Next, the computer may calculate the areas of the identified nucleus and cytoplasm (S720).

Next, the computer may calculate an HSIL score of each of the identified cells on the basis of the ratio between the areas of the cell nucleus and cytoplasm (S730).

For example, a probability of the HSIL category may be calculated on the basis of a value obtained by dividing the area of the nucleus by the area of the cytoplasm but embodiments are not limited thereto.

As described above, in order to identify the areas of the nucleus and the cytoplasm and accurately calculate the areas thereof, different pre-processing methods such as color adjustment may be performed in the calculation of the areas but embodiments are not limited thereto.

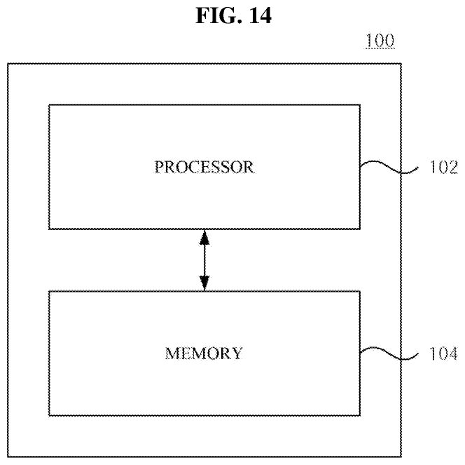

FIG. 14 is a block diagram of an apparatus according to an embodiment.

A processor 102 may include one or more cores (not shown), a graphics processor (not shown) and/or and a connection path (e.g., a bus or the like) for transmitting signals to and receiving signals from other components

In one embodiment, the processor 102 executes one or more instructions stored in a memory 104 to perform the methods described above with reference to FIGS. 1 to 13.

The processor 102 may further include a random access memory (RAM) (not shown) and a read-only memory (ROM) (not shown) for temporarily and/or permanently storing signals (or data) processed by the processor 102. The processor 102 may be embodied as a system-on-chip (SoC) including at least one of a graphic processor, a RAM, or a ROM.

The memory 104 may store programs (one or more instructions) for processing and controlling of the processor 102. Programs stored in the memory 104 may be divided into a plurality of modules according to functions.

The operations of the methods or algorithm described above in connection with embodiments of the present disclosure may be implemented directly by hardware, a software module executed by hardware, or a combination thereof. The software module may be installed in a RAM, a ROM, an erasable programmable ROM (EPROM), an electrically erasable programmable ROM (EEPROM), a flash memory, a hard disk, a removable disk, a CD-ROM, or any form of computer-readable recording medium well known in the technical field to the present disclosure pertains.

Components of the present disclosure may be embodied in the form of a program (or an application) and stored in a medium to be executed in combination with a computer which is hardware. The components of the present disclosure may be implemented by software programming or software elements, and similarly, embodiments may be implemented in a programming or scripting language such as C, C++, Java, or an assembler, including data structures, processes, routines, or various algorithms which are combinations of other programming components. Functional aspects may be implemented by an algorithm executed by one or more processors.

While embodiments of the present disclosure have been described above with reference to the accompanying drawings, it will be obvious to those of ordinary skill in the art that the present disclosure may be embodied in many different forms without departing from the technical spirit or essential features thereof. Therefore, it should be understood that the embodiments described above are merely examples in all respects and not restrictive.

1. A method of diagnosing cervical cancer using an artificial intelligence-based medical image analysis, which is performed by a computer, the method comprising:
obtaining a captured image of cervical cells of an object;
pre-processing the image;
identifying one or more cells in the pre-processed image;
determining whether the identified one or more cells are normal; and
diagnosing whether the object has cervical cancer on the basis of a result of determining whether the identified one or more cells are normal.

2. The method of claim 1, wherein the identifying of one or more cells in the pre-processed image and the determining of whether the identified one or more cells are normal comprise identifying one or more cells in the pre-processed image using a previously learned artificial intelligence model and determining whether the identified one or more cells are normal.

3. The method of claim 2, further comprising:
obtaining training data including one or more cervical cell images;
pre-processing images included in the training data; and
training the artificial intelligence model using the images pre-processed in the pre-processing of the images included in the training data.

4. The method of claim 3, wherein the pre-processing of the images included in the training data comprises, for each of the images included in the training data:
resizing the image;
adjusting a color of the resized image;
deriving a contour of the color-adjusted image; and
cropping the image on the basis of the derived contours.

5. The method of claim 3, wherein the training of the artificial intelligence model comprises:
obtaining a pre-processed high-resolution image and a pre-processed low-resolution image;
training a first model using the high-resolution image;
training a second model using the low-resolution image; and
assembling results of training the first model and the second model.

6. The method of claim 1, wherein the obtaining of the captured image of the cervical cells of the object comprises:
determining suitability of the obtained image; and
requesting to obtain an image again on the basis of the determined suitability,
wherein the requesting of the obtaining of an image again comprises at least one of requesting to capture an image again, and requesting to obtain a sample again.

7. The method of claim 1, wherein the determining of whether the identified one or more cells are normal comprises classifying the identified one or more cells into at least one of categories including normal, Atypical Squamous Cells of Undetermined Significance (ASCUS), Atypical Squamous Cells, cannot exclude HSIL (ASCH), Low-grade Squamous Intraepithelial Lesions (LSIL), High-grade Squamous Intraepithelial Lesions (HSIL), and a cancer, and
the diagnosing of whether the object has cervical cancer comprises:
counting the number of cells classified into each of the categories in the classifying of the identified one or more cells;
assigning a weight to each of the categories;
calculating a cervical cancer diagnosis score on the basis of the weight and the number of counted cells for each of the categories; and
diagnosing whether the object has cervical cancer on the basis of the calculated diagnosis score.

8. The method of claim 7, wherein the classifying of the identified one or more cells comprises:
identifying a nucleus and cytoplasm of each of the identified one or more cells;
calculating areas of the identified nucleus and cytoplasm; and
calculating an HSIL score of each of the identified one or more cells on the basis of a ratio between the areas of the nucleus and cytoplasm.

9. An apparatus comprising:
a memory storing one or more instructions; and
a processor configured to execute the one or more instructions stored in the memory,
wherein the processor executes the one or more instructions to perform the method of claim 1.

10. A computer program stored in a computer-readable recording medium to perform the method of claim 1 when connected to a computer which is hardware.
